(19) United States
(12) Patent Application Publication
(10) Pub. No.: US 2025/0248429 A1
(43) Pub. Date: Aug. 7, 2025

(54) SYSTEMS AND METHODS OF PREPARING CULTIVATED MEAT AND MEAT ANALOGS FROM PLANT-BASED PROTEINS

(71) Applicant: Omeat Inc., Los Angeles, CA (US)

(72) Inventors: Alireza Khademhosseini, Los Angeles, CA (US); Seyedvahid Hosseini, Newbury Park, CA (US)

(73) Assignee: Omeat Inc., Los Angeles, CA (US)

(21) Appl. No.: 18/855,726

(22) PCT Filed: Apr. 14, 2023
(86) PCT No.: PCT/US2023/018664

§ 371 Date: Oct. 10, 2024

Related U.S. Application Data
(60) Provisional application No. 63/330,814, filed on Apr. 14, 2022.
(60) Provisional application No. 63/337,554, filed on May 2, 2022.

(51) Int. Cl. A23L 13/40

(52) U.S. Cl. CPC A23L 13/426; A23L 13/424

(57) ABSTRACT

The present disclosure generally relates to cultivated meat and other animal-derived products comprising plant proteins. For example, some aspects are generally directed to products comprising texturized plant protein as well as animal-derived components, such as blood plasma or red blood cell lysates. These may be useful, for example, to provide color, taste, nutrients, etc. to the products, e.g., that are not readily achievable with pure texturized plant proteins. In addition, in some cases, such animal-derived components may be sustainably harvested, e.g., by repeatedly extracting blood components from non-human animals in a manner which does not kill the animals. Other aspects are generally directed to methods of making or using such cultivated meat products, kits involving these, or the like.

### RELATED APPLICATIONS

This application claims the benefit of U.S. Provisional Patent Application Ser. No. 63/337,554, filed May 2, 2022, entitled “Systems and Methods of Preparing Cultivated Meat and Meat Analogs from Plant-Based Proteins,” and of U.S. Provisional Patent Application Ser. No. 63/330,814, filed Apr. 14, 2022, entitled “Systems and Methods for Protein Recovery from Cell Culture Media,” each incorporated herein by reference in its entirety.

### FIELD

The present disclosure generally relates to cultivated meat and other animal-derived products comprising plant proteins.

### BACKGROUND

Texturized vegetable proteins have been known since the 1960's as a meat substitute. They are typically formed from certain plant proteins such as soy proteins, pea proteins, cottonseed proteins, wheat proteins, or oat proteins. They can be extruded into various shapes (e.g., chunks, flakes, nuggets, grains, and strips). After extrusion, texturized vegetable proteins typically have a fibrous, spongy matrix, similar in texture to meat. Accordingly, it is often used as a meat substitute in various foods and recipes. Such texturized vegetable proteins are sold in grocery stores by a variety of different manufacturers and under a variety of brand names. However, despite being a common ingredient found in many foods, such texturized vegetable proteins have various drawbacks, such as diminished flavor or lack of nutritional value.

### SUMMARY

The present disclosure generally relates to cultivated meat and other animal-derived products comprising plant proteins. The subject matter of the present disclosure involves, in some cases, interrelated products, alternative solutions to a particular problem, and/or a plurality of different uses of one or more systems and/or articles.

One aspect is generally directed to a meat product. According to one set of embodiments, the meat product comprises a texturized plant protein, non-human blood plasma, and a lysate of non-human red blood cells.

In another set of embodiments, the meat product comprises a ground beef replica comprising a texturized plant protein, non-human muscle cells, non-human blood plasma, and a lysate of non-human red blood cells.

The meat product, in yet another set of embodiments, comprises a texturized plant protein, and non-human blood plasma. In another set of embodiments, the meat product comprises a texturized plant protein, and a fat replica comprising a fat emulsion and non-human blood plasma.

In one set of embodiments, the meat product comprises a texturized plant protein, microcarriers comprising fibrin, and non-human cells on the microcarriers. In another set of embodiments, the meat product comprises a texturized plant protein, and a muscle replica comprising microcarriers and non-human muscle cells. In yet another set of embodiments, the meat product comprises a texturized plant protein, and a hydrogel comprising crosslinked non-human blood plasma.

The meat product, in one set of embodiments, comprises a texturized plant protein, and non-human blood. In another set of embodiments, the meat product comprises a texturized plant protein, and non-human red blood cells. In yet another set of embodiments, the meat product comprises a texturized plant protein, and non-human blood plasma. The meat product, in still another set of embodiments, comprises a texturized plant protein, non-human blood plasma, and non-human red blood cells.

Another aspect is generally drawn to a method. According to one set of embodiments, the method includes mixing a texturized plant protein, non-human blood plasma, and a lysate of non-human red blood cells to produce a meat product.

The method, in another set of embodiments, includes withdrawing a blood sample from a non-human animal, processing the blood sample to produce non-human blood plasma and a lysate of non-human red blood cells, and producing a product comprising a mixture of the non-human blood plasma, the lysate of non-human red blood cells, and texturized plant protein.

In still another set of embodiments, the method comprises mixing a texturized plant protein and non-human blood plasma to produce a product. According to yet another set of embodiments, the method comprises mixing a texturized plant protein and a lysate of non-human red blood cells to produce a product.

In another aspect, the present disclosure encompasses methods of making one or more of the embodiments described herein, for example, a cultivated meat product or other animal-derived product. In still another aspect, the present disclosure encompasses methods of using one or more of the embodiments described herein, for example, a cultivated meat product or other animal-derived product.

Other advantages and novel features of the present disclosure will become apparent from the following detailed description of various non-limiting embodiments of the disclosure.

### DETAILED DESCRIPTION

The present disclosure generally relates to cultivated meat and other animal-derived products comprising plant proteins. For example, some aspects are generally directed to products comprising texturized plant protein as well as animal-derived components, such as blood plasma or red blood cell lysates. These may be useful, for example, to provide color, taste, nutrients, etc. to the products, e.g., that are not readily achievable with pure texturized plant proteins. In addition, in some cases, such animal-derived components may be sustainably harvested, e.g., by repeatedly extracting blood components from non-human animals in a manner which does not kill the animals. Other aspects are generally directed to methods of making or using such meat products, kits involving these, or the like.

Certain aspects as discussed herein are generally directed to meat products that contain texturized plant protein and components derived from the blood of animals, such as blood plasma or cellular lysates. While other meat products may contain texturized plant proteins, in most cases, those meat products are intended to be animal-free, and thus do not contain any components take from animals. Thus, such meat products are often sold to vegetarian or vegan consumers, as no part of those meat products contains anything that is derived from an animal.

In contrast, various meat products as discussed herein may contain animal-derived components. A variety of such animal-derived components may be present. For example, components derived from the blood of animals may be present. A specific non-limiting example is blood plasma, which may be harvested from an animal using one or more blood draws. The blood may be processed to obtain the plasma, e.g., using plasma apheresis or other suitable techniques. As another example, a lysate may be obtained from red blood cells taken from the blood. The lysate may be obtained, for instance, by exposing blood cells to hypoosmotic or distilled water. As yet another example, a fat emulsion may be prepared by emulsifying fat with blood plasma. For example, fibrin within the plasma may be caused to clot and/or by causing the fibrin to crosslink, e.g., by exposing it to thrombin, calcium, or other clotting agents such as those described herein. As still another example, the meat product may contain microcarriers, e.g., prepared from fibrin obtain from the blood of an animal. In addition, more than one of these and/or other animal-derived components may also be present, in yet other examples.

It should be noted that in some embodiments, the blood or other animal-derived components may be obtained without slaughtering the animal, e.g., on a periodic basis. Thus, certain embodiments as discussed herein are directed toward repeated blood collection, for example, to obtain whole blood or blood components, etc., that can be used within cultivated meat products or other animal-derived products for example, in conjunction with texturized plant proteins. Accordingly, some embodiments as discussed herein are directed to meat products that include texturized plant proteins as well as components derived from animals; these may not necessarily be animal-free products, although they may be prepared without necessarily killing the animal.

Thus, various aspects are described herein that are directed to cultivated meat and other animal-derived products comprising plant proteins, such as texturized plant protein, and certain animal-derived components, such as blood plasma or cell lysates, as well as various systems and methods for producing such products.

For example, certain aspects are generally directed to meat products that contain texturized plant proteins. As is known to those of ordinary skill in the art, texturized plant proteins are materials derived from certain plants that have been processed or “texturized” from a flour-type material into a material which has a more meat-like texture, typically using processes such as extrusion or fiber spinning. Such texturized plant proteins can be used in food, e.g., as a meat substitute, for instance, since such texturized plant proteins can be processed to produce a product having a certain chewiness or fibrous character. In some cases, for example, the texturized plant proteins may be processed such that the final product is able to withstand hydration, cooking, or other common practices used in preparing the food.

In some embodiments, at least 10 wt %, at least 20 wt %, at least 30 wt %, at least 40 wt %, at least 50 wt %, at least 55 wt %, at least 60 wt %, at least 65 wt %, at least 70 wt %, at least 75 wt %, at least 80 wt %, at least 85 wt %, at least 90 wt %, etc. of the product may comprise texturized plant protein. In addition, in some embodiments, no more than 95 wt %, no more than 90 wt %, no more than 85 wt %, no more than 80 wt %, no more than 75 wt %, no more than 70 wt %, no more than 65 wt %, no more than 60 wt %, no more than 55 wt %, no more than 50 wt %, no more than 40 wt %, no more than 30 wt %, no more than 20 wt %, or no more than 10 wt % of the product may comprise texturized plant protein. Combinations of any of these are also possible in certain embodiments. For example, the product may have between 60 wt % and 80 wt %, between 70 wt % and 95 wt %, between 50 wt % and 65 wt %, between 75 wt % and 80 wt %, etc. of texturized plant protein.

Texturized plant proteins can be prepared from a variety of different edible protein sources. One of the more common plants that can be used is soy. However, texturized plant proteins can also be made from other plants, such as pea, chickpea, cottonseed, corn, wheat, peanut, lentil, mycoproteins (e.g., from mushrooms, wood ears, or other edible fungi), quinoa, wheat, oat, or the like.

A variety of methods may be used to form a texturized plant protein. Two of the more common methods for texturizing plant proteins are extrusion and fiber spinning. In one example of an extrusion process, a plant-based flour or protein concentrate may be used as a starting material. This can be mixed with water, salt, colors, flavors, seasonings, nutritional additives, and/or other ingredients, then heated and passed through an extruder. The product expands as it leaves the extruder to form a texturized plant protein, and may be extruded to from various shapes (e.g., chunks, flakes, nuggets, grains, strips, or the like). The size and shape of extruded material can be controlled, for example, by controlling the temperature, the flow rate through the extruder, the size and speed of cutting of the extruded protein as it passes through the extruder, or the like.

In one example of a fiber spinning process, plant proteins (e.g., from a plant-based flour or a protein concentrate) may be solubilized, e.g., in an alkaline medium. The protein solution may be passed through a spinneret to form fibers, which can be coagulated in an acidic bath. In some cases, water, salt, colors, flavors, seasonings, nutritional additives, and/or other ingredients may be added. The fibers can be prepared, for example, by stretching them over a series of rolls, to form the texturized plant protein. In some cases, binders such as albumin can also be added to hold bundles of fibers together.

As discussed, in various aspects, the texturized plant proteins may be combined with other components, e.g., to form a cultivated meat product, or other animal-derived products such as described herein. Cultivated meat is often described using terms such as cultivated meat, cultured meat, tissue mass, cellular (or cell-based) meat, slaughter-free meat, and synthetic meat, among other related terms. Cultivated meat products may be produced using in vitro cell culture or bioreactors, as opposed to “regular” meat that is grown and harvested from live animals.

However, it should be understood that the invention is not limited to only cultivated meat products. In some cases, other animal-derived products, such as those described herein, may be prepared from animal-derived cells, but the product is not necessarily one that is intended to be eaten. For instance, cells from an animal may be cultured to form various organs that can be harvested, such as skin, hair, fur, or the like. Thus, as non-limiting examples, leather, cultivated fur, etc. can be formed, for example, by growing cells in culture such as discussed herein, without the traditional method of sacrificing animals to harvest their skin or other organs.

As mentioned, in some embodiments, products such as those described herein may include certain animal-derived components, for example, components that are derived from the blood of an animal. Thus, certain embodiments as discussed herein are not animal-free, but in fact incorporate components that derive from animals. However, in some embodiments, the blood or other animal-derived components may be obtained without killing or slaughtering the animal, e.g., on a periodic basis.

The animal-derived components may be used directly in a product such as discussed herein, or in some cases, such components may be cultured or processed before use, e.g., within a product. For instance, in some cases, animal-derived components may be produced using cells taken from an animal, but then the cells are cultured in vitro, e.g., using bioreactors, flasks, petri dishes, microwell plates, or other cell culture systems. Many cell culture systems will be known to those of ordinary skill in the art. This is in stark contrast to traditional techniques of sacrificing animals and harvesting their meat or other organs (e.g., skin, internal organs, etc.) for food or other uses. Although the original cells seeded to form the product may have originated or otherwise have originally been derived from a living animal, the bulk of the cells forming the actual product were grown or cultured in an in vitro setting, rather than naturally as part a living animal.

A variety of products may be formed from cells cultured in vitro. For instance, in certain embodiments, the products may form “cultivated meat,” or meat that is intended to be eaten, for example, by humans. It will be appreciated that, because it is to be eaten, such products will often be formed of edible or digestible materials, e.g., materials that can be digested, or degraded to form generally nontoxic materials within the digestive system. For instance, the product contain animal-derived cells (e.g., derived from a chicken, a cow, a pig, a sheep, a goat, a deer, a fish, a duck, a turkey, a shrimp, or other animals that are commonly recognized for widespread human consumption), such as fat cells, muscle cells, or the like. The cells may be wild-type or naturally-occurring cells (e.g., harvested from an animal), although in some embodiments, the cells may include genetically engineered cells, e.g., engineered in a way to increase proliferation.

In addition, in some embodiments, the cultivated meat product or other animal-derived product may contain other edible materials, such as plant-originated materials, including texturized plant proteins. Additional non-limiting examples of edible materials include proteins, carbohydrates, sugars, saccharides, plant-based fats, etc., as well as polymers formed from these (for example, polylactic acid, polyglycolic acid, cellulose, etc.). In some cases, the edible materials may be digested to form nutrients, e.g., such as amino acids, sugars, etc. that have nutritional value, for example, when taken up into the body. However, in some cases, the edible materials cannot be digested, and/or can be digested to form non-nutrients that cannot be absorbed as nutrients, but can be passed through the digestive system without detrimental effects.

For example, in one set of embodiments, the product may contain blood components such as blood plasma. As mentioned, such blood components may be taken from animals, and in some cases, without killing the animal. For instance, in some embodiments, blood components may be harvested from an animal by apheresis or other methods, for example, by using gravity to separate cells, or by using techniques such as centrifugation or sedimentation of red blood cells at atmospheric or high pressure. In some embodiments, blood cells may be removed from a living animal, and plasma and other blood components including platelets, red blood cells, fibrin and other proteins, plasma soluble factors such as fibrin, albumin, minerals, vitamins, growth factors, etc. may be removed, e.g., using apheresis. In some cases, the remaining blood product may be returned to the animal. Such blood components can thereby be obtained from living animals without killing the animals. See, e.g., Int. Pat. Apl. Ser. No. PCT/US22/19631, entitled “Methods and Systems of Producing Products Such as Animal Derived Products,” filed on Mar. 9, 2022, the disclosure of which is hereby incorporated by reference. In some embodiments, any desired blood component can be harvested including plasma (e.g., platelet rich plasma or PRP), platelets, etc.

In one set of embodiments, plasma can be derived from whole blood, e.g., from which red blood cells have been removed, such as by centrifugation. Plasma can also be derived from whole blood by, for example, using apheresis or by removing platelets from the platelet-rich plasma product using, for example, centrifugation or other suitable techniques. Plasma may contain a variety of growth factors that are in the blood such as transforming growth factor beta, fibroblast growth factor, insulin-like growth factor 1, insulin-like growth factor 2, vascular endothelial growth factor, epidermal growth factor, interleukin 8, keratinocyte growth factor and connective tissue growth factor. In addition, lysing the platelets in the platelet-rich plasma can release platelet-derived growth factor, which can be used in certain embodiments.

Plasma may contain growth factors and other solutes. For example, plasma may comprise adhesive proteins, e.g., fibrinogen, which can facilitate non-human cells, e.g., myoblasts, to adhere to microcarriers, e.g., fibrin microcarriers. In some cases, the plasma may comprise one or more growth factors, e.g., platelet-derived growth factor. Other example solutes include dissolved proteins (e.g., 6-8% by weight), e.g., serum albumins, goblins, and fibrinogen), glucose, clotting factors, electrolytes (Na⁺, Ca²⁺, Mg²⁺, HCO₃⁻, Cl⁻, etc.), hormones, etc.

In some embodiments, at least 1 wt %, at least 3 wt %, at least 5 wt %, at least 10 wt %, at least 15 wt %, at least 20 wt %, at least 25%, at least 30 wt %, at least 40 wt %, at least 50 wt %, etc. of the product comprises blood plasma. In addition, in certain embodiments, no more than 50 wt %, no more than 40 wt %, no more than 30 wt %, no more than 25 wt %, no more than 20 wt %, no more than 15 wt %, no more than 10 wt %, no more than 5 wt %, no more than 3 wt %, or no more than 1 wt % of the product comprises a blood plasma. Combinations of any of these are also possible in certain cases; for example, the product may have between 5 wt % and 10 wt %, between 10 wt % and 25 wt %, between 1 wt % and 20 wt %, between 15 wt % and 25 wt %, etc. of blood plasma.

As another example, in certain embodiments, the product may include a cell lysate. In some cases, the cell lysate may contain heme. The heme may be used as a colorant in the meat product. In one embodiment, the heme may be obtained from non-human red blood cells. For example, red blood cells may be lysed, e.g., by exposing the cells to hypoosmotic or distilled water to form a lysate of non-human red blood cells. The red blood cells may contain hemoglobin, a protein structurally similar to myoglobin. Hemoglobin also may contain a heme moiety. Lysing the red blood cells may release hemoglobin, e.g., into solution. Furthermore, in addition to these, other methods of lysing red blood cells can be used in other embodiments, including those discussed in more detail herein. Other non-limiting examples of lysates of non-human red blood cells may be found in Int. Pat. Apl. Ser. No. PCT/US22/19615, entitled “Production of Heme for Cell-Based Meat Products,” filed on Mar. 9, 2022, incorporated herein by reference in its entirety.

Thus, certain embodiments are generally directed to products containing lysates, e.g., obtained from non-human red blood cells. Such a lysate may be produced, for example, by lysing red blood cells, using any of a variety of lysing techniques. For example, the non-human red blood may arise from a chicken, a cow, a pig, a sheep, a goat, a deer, a fish, a duck, a turkey, a shrimp, or other animals such as those described herein.

For example, in one set of embodiments, cells may be lysed by exposing them to hypoosmotic water, such as distilled water. Cells typically have an osmolarity of about 300 mOsm; hypoosmotic solutions with osmolarities less than this (including distilled water, having an osmolarity of approximately zero) may cause water to enter the cells, ultimately causing them to burst or lyse. Accordingly, in certain embodiments, red blood cells are exposed to a hypoosmotic solution to cause the cells to lyse. In some embodiments the cells may be exposed to a volume of hypoosmotic water that is at least sufficient to lyse the red blood cells. For example, the volume may be at least 5%, at least 10%, at least 20%, at least 30%, at least 40%, or at least 50% of the volume of the solution containing the cells. These precents are percent by volume.

In addition, other methods of lysing red blood cells can be used in other embodiments. As one non-limiting example, acoustic energy may be used to lyse the cells. For example, the cells may be exposed to a sonicator or an ultrasonic bath to cause the cells to lyse. As yet another non-limiting example, the cells may be exposed to one or more freeze-thaw cycles, e.g., where the temperature is lowered to below the freezing point of the solution, thereby causing ice to form, killing at least some of the cells.

In certain embodiments, the cells may be lysed such that at least 50% of the cells have been lysed or ruptured, and in some embodiments, such that at least 60%, at least 70%, at least 80%, at least 85%, at least 90%, at least 95%, or at least 99% of the cells have been lysed or ruptured. One or more than one lysing technique may be used in various embodiments.

In some embodiments, at least 1 wt %, at least 3 wt %, at least 5 wt %, at least 10 wt %, at least 15 wt %, at least 20 wt %, at least 25%, at least 30 wt %, at least 40 wt %, at least 50 wt %, etc. of the product comprises a lysate. In addition, in certain embodiments, no more than 50 wt %, no more than 40 wt %, no more than 30 wt %, no more than 25 wt %, no more than 20 wt %, no more than 15 wt %, no more than 10 wt %, no more than 5 wt %, no more than 3 wt %, or no more than 1 wt % of the product comprises a lysate. Combinations of any of these are also possible in certain cases; for example, the product may have between 5 wt % and 10 wt %, between 10 wt % and 25 wt %, between 1 wt % and 20 wt %, between 1 wt % and 5 wt %, etc. of a lysate.

In another example, in certain embodiments, the product may include blood or a blood product, for example, whole blood or red blood cells. These may not necessarily be lysed. In some cases, the blood may arise from a non-human animal, e.g., as discussed herein. In some embodiments, at least 1 wt %, at least 3 wt %, at least 5 wt %, at least 10 wt %, at least 15 wt %, at least 20 wt %, at least 25%, at least 30 wt %, at least 40 wt %, at least 50 wt %, etc. of the product comprises whole blood or a blood product, such as red blood cells. In addition, in certain embodiments, no more than 50 wt %, no more than 40 wt %, no more than 30 wt %, no more than 25 wt %, no more than 20 wt %, no more than 15 wt %, no more than 10 wt %, no more than 5 wt %, no more than 3 wt %, or no more than 1 wt % of the product comprises whole blood or a blood product, such as red blood cells. Combinations of any of these are also possible in certain cases; for example, the product may have between 5 wt % and 10 wt %, between 10 wt % and 25 wt %, between 1 wt % and 20 wt %, between 1 wt % and 5 wt %, etc.

As yet another example, in certain embodiments, the product may include a muscle replica. The muscle replica may comprise, for example, non-human muscle cells. In some cases, the cells may be present on microcarriers or scaffolds, e.g., as discussed herein. For instance, in some embodiments, a muscle replica may be formed by seeding myoblasts on microcarriers or scaffolds, then growing the cells within a bioreactor to form the muscle replica. Non-limiting examples of muscle replicas may be found in Int. Pat. Apl. Ser. No. PCT/US22/19601, entitled “Methods and Systems of Preparing Cultivated Meat from Blood or Cellular Biomass,” filed on Mar. 9, 2022, incorporated herein by reference in its entirety. In addition, in some cases, the microcarriers or scaffolds may have structures, such as grooves, that may allow the cells such as myoblasts to become aligned in a specific direction, although this is not a requirement. Such structures are described in Int. Pat. Apl. Ser. No. PCT/US22/19590, entitled “Constructs Comprising Fibrin or Other Blood Products for Meat Cultivation and Other Applications,” filed on Mar. 9, 2022, incorporated herein by reference in its entirety.

In some embodiments, at least 1 wt %, at least 3 wt %, at least 5 wt %, at least 10 wt %, at least 15 wt %, at least 20 wt %, at least 25%, at least 30 wt %, at least 40 wt %, at least 50 wt %, etc. of the product comprises a muscle replica. In addition, in certain embodiments, no more than 50 wt %, no more than 40 wt %, no more than 30 wt %, no more than 25 wt %, no more than 20 wt %, no more than 15 wt %, no more than 10 wt %, no more than 5 wt %, no more than 3 wt %, or no more than 1 wt % of the product comprises a muscle replica. Combinations of any of these are also possible in certain cases; for example, the product may have between 5 wt % and 10 wt %, between 10 wt % and 25 wt %, between 1 wt % and 20 wt %, etc. of a muscle replica.

A variety of techniques may be used to grow cells within the bioreactor or other cell culture system, e.g., to form a muscle replica, or other components such as any of those described herein. For instance, the cells may be grown at body temperature (e.g., about 38.5° C. for cow cells, about 41° C. for chicken cells, about 39-40° C. for pig cells, about 40-42° C. for duck cells, etc.). In some embodiments, during cultivation, the cells may have a shear stress applied to them of at least 0.005 newton/meter squared, of at least 0.1 newton/meter squared, of at least 0.2 newton/meter squared, of at least 0.3 newton/meter squared, of at least 0.4 newton/meter squared, of at least 0.5 newton/meter squared, of at least 0.6 newton/meter squared, of at least 0.7 newton/meter squared, of at least 0.8 newton/meter squared, etc.

In some embodiments, the product may include a microcarrier or a scaffold. In certain embodiments, one or more cells, such as muscle cells, may be seeded on the microcarriers or scaffolds, e.g., as described herein. The microcarriers or scaffolds may comprise any material that forms an edible hydrogel, such as fibrin. For example, in one embodiment, a microcarrier may be formed from a non-human blood plasma which contains plasma-rich fibrinogen that can be crosslinked or otherwise processed to form a fibrin hydrogel. Such crosslinking can be achieved by exposure to thrombin, calcium, or other conditions such as those described herein. In some embodiments, fibrin hydrogels are formed using non-human blood plasma containing fibrinogen, e.g., at least 10 wt %, or more in some cases. Non-limiting examples of microcarriers include those disclosed in Int. Pat. Apl. Ser. No. PCT/US22/19590, entitled “Constructs for Meat Cultivation and Other Applications,” filed on Mar. 9, 2022, incorporated herein by reference in its entirety.

Thus, some embodiments, such as described herein, are generally directed to microcarriers or scaffolds comprising fibrin. These may be present in a product, in accordance with certain embodiments. Fibrin is an edible fibrous protein involved in the clotting of blood. It can be formed, for example, by the action of the protease inhibitor thrombin on fibrinogen, which can cause it to polymerize and form a clot. Fibrin can be used as a passive scaffolding material in some embodiments. However, in some embodiments, fibrin can specifically bind certain growth factors in the cell culture media that promote cell adhesion, proliferation, and migration. Non-limiting examples include fibronectin, hyaluronic acid, von Willebrand factor, or the like.

In certain embodiments, microcarriers or scaffolds such as those discussed herein may be treated to facilitate binding of cells, such as myoblasts. For example, the microcarriers or scaffolds may be exposed to non-human serum, which may include growth factors that bind to the microcarriers or scaffolds. The growth factors may, for example, promote cell adhesion, proliferation, and/or migration of cells into the microcarriers or scaffolds. In addition, in some cases, the microcarriers or scaffolds may have structures, such as grooves, that may allow the cells such as myoblasts to become aligned in a specific direction, although this is not a requirement. Such structures are described in, for example, Int. Pat. Apl. Ser. No. PCT/US22/19590, entitled “Constructs Comprising Fibrin or Other Blood Products for Meat Cultivation and Other Applications,” filed on Mar. 9, 2022, incorporated herein by reference in its entirety.

In some embodiments, the microcarriers or scaffolds may comprise any material that forms an edible hydrogel, such as fibrin. For example, in one embodiment, a microcarrier may be formed from a non-human blood plasma which contains plasma-rich fibrinogen that can be crosslinked or otherwise processed to form a fibrin hydrogel. Such crosslinking can be achieved by exposure to thrombin, calcium, or other conditions such as those described herein. In some embodiments, fibrin hydrogels are formed using non-human blood plasma containing fibrinogen, e.g., at least 10 wt %, or more in some cases.

As mentioned, in certain embodiments, non-human cells such as myoblasts may be seeded on the microcarriers or other scaffolds, and in some cases grown in a bioreactor or other in vitro cell culture system. For instance, myoblasts may be grown on microcarriers and, in some embodiments, allowed to differentiate or fuse to form aligned myotubes, e.g., within a bioreactor or other system.

In one set of embodiments, cells are seeded onto microcarriers or other scaffolds. A scaffold may define a substrate that the cells are able to divide and proliferate on, e.g., forming tissue that forms the basis of the meat product. A variety of cell scaffold structures can be used, including scaffolds known by those of ordinary skill in the art. The scaffold may thus have any suitable size or shape. In some cases, the scaffold may be anisotropic, i.e., not exhibiting radial or spherical symmetry. In addition, in certain embodiments, the scaffold may be relatively solid, or have holes or pores. Thus, for example, the scaffold may have any suitable degree of porosity. One or more than one scaffold may be present. For instance, in certain cases, the scaffold comprises one or a plurality of microcarriers, e.g., as described herein. If more than one scaffold is present, the scaffolds may be independently the same or different. In addition, in certain embodiments, the scaffold may have one or more grooves, e.g., as discussed herein.

In one set of embodiments, a microcarrier or a scaffold may have a largest or maximum internal dimension of less than 100 mm, less than 80 mm, less than 70 mm, less than 60 mm, less than 50 mm, less than 40 mm, less than 30 mm, less than 20 mm, less than 10 mm, less than 5 mm, less than 3 mm, less than 2 mm, or less than 1 mm. In addition, in some cases, a microcarrier or a scaffold may have a maximum internal dimension that is at least 1 mm, at least 2 mm, at least 3 mm, at least 5 mm, at least 10 mm, at least 20 mm, at least 30 mm, at least 40 mm, at least 50 mm, at least 60 mm, at least 70 mm, at least 80 mm, at least 90 mm, at least 100 mm, etc. Combinations of any of these dimensions are also possible in some embodiments.

The microcarrier or scaffold may comprise any suitable material. For example, in one set of embodiments, the microcarrier or scaffold may comprise fibrin, or another edible material, including any of those described herein. This may be useful for products such as cultivated meat, where the animal-derived product will be eaten, e.g., by humans or other animals. In some embodiments, the microcarrier or scaffold may comprise a hydrogel, e.g., a fibrin hydrogel, or other hydrogels such as those described herein.

In some cases, at least 50 wt %, at least 60 wt %, at least 70 wt %, at least 80 wt %, at least 90 wt %, or substantially all of a scaffold is formed from fibrin, and/or another edible material. The fibrin may arise from any suitable source. For example, the fibrin may arise from a non-human animal, such as a non-human mammal. Non-limiting examples include cows, pigs, sheep, goats, or the like. In some cases, the fibrin may arise from the blood of such an animal. For instance, in some embodiments, the fibrin may be prepared by acquiring blood or blood plasma from an animal, and processing it to produce fibrin. For example, in one set of embodiments, the blood is exposed to a protease inhibitor such as thrombin, which may cause fibrinogen to clot to form fibrin. The fibrin may be harvested, and used as discussed herein, e.g., to produce scaffolds such as microcarriers. In addition, in some cases, fibrin may be obtained from fibrinogen, which may be bought commercially, obtained from blood plasma, or the like.

In some cases, the blood may be acquired from the animal without killing the animal. For instance, blood may be withdrawn from the animal at spaced intervals, so as to allow the animal time to recover and produce new blood. For instance, blood may be withdrawn from the animal every 4 weeks, every 6 weeks, every 2 months, or the like, e.g., as discussed herein.

The fibrin may be processed to form a scaffold. In one set of embodiments, the scaffold may take the form of one or more microcarriers. The microcarriers may have any shape or size. In some cases, more than one type of microcarrier may be present, e.g., some of which may have various materials, shapes, sizes, etc., such as are described herein. For example, in some embodiments, at least some of the microcarriers may be substantially spherical or exhibit spherical symmetry, although in other embodiments, at least some of the microcarriers may be non-spherically symmetric (for example, triangular) or may be anisotropic. In addition, in certain cases, at least some of the microcarriers may have a plurality of grooves, e.g., as discussed herein.

In some cases, however, some or all of the microcarriers may not necessarily be spherical. For example, at least some of the microcarriers may have shapes such as cubical, rectangular solid, triangular, tetrahedral, octahedral, irregular, etc. In some cases, at least some of the microcarriers have a shape that is substantially planar. For instance, the microcarrier may have a generally rectangular shape where the smallest dimension of the rectangular solid is substantially smaller than either of the other two dimensions, for example, by a factor of at least 3, at least 5, or at least 10, etc.

In addition, in certain cases, at least some of the microcarriers have a relatively large surface to volume ratio. This may be important, for example, in embodiments where the microcarriers contain a plurality of grooves, e.g., as discussed herein. In contrast, a perfect sphere would have the smallest possible surface to volume ratio for a given volume of material. As a nonlimiting example, the surface to volume ratio may be at least 100, at least 200, at least 300, etc., e.g., for a sheet thickness of 0.01 mm surface and an area of 1 mm×10 mm.

In some embodiments, at least some of the microcarriers or other types of scaffolds may have one or more grooves defined therein. (However, it should be understood that such grooves are not always required in other embodiments.) Examples of such grooves include those discussed in Int. Pat. Apl. Ser. No. PCT/US22/19590, entitled “Constructs Comprising Fibrin or Other Blood Products for Meat Cultivation and Other Applications,” filed on Mar. 9, 2022, incorporated herein by reference in its entirety. In some embodiments, the grooves may be positioned or sized within the microcarriers or other scaffolds to allow the myoblasts to be directional or aligned, e.g., to allow them to fuse together to become myotubes. One or more grooves may be present. For instance, a microcarrier or other scaffold may have at least 2, at least 3, at least 4, at least 5, at least 7, at least 10, at least 12, at least 15, at least 20, at least 25, at least 30, at least 40, at least 50, at least 70, or at least 100 or more grooves defined therein. If more than one type of microcarrier or scaffold is present, they may independently have the same or different numbers of grooves. In some cases, the average number of grooves present within the microcarriers may have the ranges described here. The grooves may be positioned in any orientation on the microcarriers. For instance, the grooves may be substantially parallel to each other, e.g., to promote the formation of substantially aligned myotubes. The grooves may also have any profile, e.g., square or rectangular, and any aspect ratio (i.e., width to height).

In some embodiments, the microcarriers (or other scaffolds) may be formed to have any of a wide variety of shapes, such as flakes, plates, fibers, whiskers, or the like, e.g., having dimensions such as any of those described herein. In addition, as previously noted, in certain embodiments, some of these shapes may contain grooves.

As a non-limiting example, the microcarriers may have the form of fibers, e.g., having an average length of at least 1 micrometer, at least 2 micrometers, at least 3 micrometers, at least 4 micrometers, at least 5 micrometers, at least 10 micrometers, at least 20 micrometers, at least 30 micrometers, at least 40 micrometers, at least 50 micrometers, at least 100 micrometers, at least 200 micrometers, at least 300 micrometers, at least 400 micrometers, at least 500 micrometers, at least 1 mm, at least 2 mm, at least 3 mm, at least 4 mm, at least 5 mm, at least 1 cm, at least 2 cm, at least 5 cm, at least 10 cm, at least 20 cm, at least 30 cm, at least 50 cm, etc. In addition, in certain embodiments, the fibers may have an average length of no more than 100 cm, no more than 50 cm, no more than 30 cm, no more than 20 cm, no more than 10 cm, no more than 5 cm, no more than 4 cm, no more than 3 cm, no more than 2 cm, no more than 1 cm, no more than 5 mm, no more than 4 mm, no more than 3 mm, no more than 2 mm, no more than 1 mm, no more than 500 micrometers, no more than 400 micrometers, no more than 300 micrometers, no more than 200 micrometers, no more than 100 micrometers, etc. Combinations of any of these are also possible, e.g., the fibers may have an average length of between 200 micrometers and 500 micrometers, between 500 micrometers and 5 mm, between 300 micrometers and 1 mm, between 10 micrometers and 400 micrometers, etc.

In addition, in certain embodiments, the product may comprise a fat replica. In some cases, the fat replica may comprise fat and a hydrogel. The fat may arise from any suitable source, or more than one source in some instances. For example, the fat may arise from animal cells and/or plants. Examples of plant-based fats include vegetable oil, corn oil, and other oils such as those described herein. Animal-based fats may, in some embodiments, be formed from fat (or adipose) cells that are cultivated, for example, on microcarriers, or other suitable scaffolds. The fat replica may also include a microcarrier or other scaffold. The microcarrier or other scaffold may comprise fibrin and/or other suitable materials such as those discussed herein. In addition, in some cases, the fat cells may be grown in a reactor. As yet another example, the fat may include fat that was synthetically prepared. Other non-limiting examples of fat replicas may be found in Int. Pat. Apl. Ser. No. PCT/US22/19609, entitled “Systems and Methods of Producing Fat Tissue for Cell-Based Meat Products,” filed on Mar. 9, 2022, incorporated herein by reference in its entirety.

In some embodiments, the fat in the fat replica may be present in an emulsion. An emulsion of fat may be prepared, for example, by emulsifying fat with non-human blood plasma, e.g., as previously mentioned. In some cases, the fat may be caused to form a fat emulsion by mixing the fat with non-human blood plasma. Without wishing to be bound by any theory, it is believed that the plasma has components that can emulsify fat to form fat particles such as chylomicrons. For instance, the plasma may include proteins or surfactants that can from such fat particles. The non-human blood plasma may be treated in some embodiments to form a fat replica. For example, fibrin within the plasma may be caused to clot and/or by causing the fibrin to crosslink, e.g., by exposing it to thrombin, calcium, or other clotting agents such as those described herein.

In addition, in some embodiments, a fat replica may comprise a fat emulsion contained within a hydrogel. The hydrogel may be formed from non-human blood plasma, e.g., as discussed herein, and/or another component. Non-limiting examples of such hydrogels include alginate, gelatin, or others such as those described herein. Such hydrogels and other structures are also described in Int. Pat. Apl. Ser. No. PCT/US22/19590, entitled “Constructs Comprising Fibrin or Other Blood Products for Meat Cultivation and Other Applications,” filed on Mar. 9, 2022, incorporated herein by reference in its entirety.

In one set of embodiments, the cultivated meat or other animal-derived product may contain a fat replica. As discussed, a fat replica is not “regular” fat that is naturally grown within a live animal. Instead, the fat replica may be artificially produced through a variety of techniques. For example, in one embodiment, the fat replica comprises one or more plant-based fats. Plant-based fats can include any fat obtained or extracted from a plant, e.g., vegetable oil, sunflower seed oil, corn oil, safflower oil, oleic acid, canola oil, omega-3 fatty acids, omega-6 fatty acids, olive oil, peanut oil, palm oil, cocoa butter, coconut oil, rapeseed oil, linseed oil, almond oil, sesame oil, soybean oil, etc. As another example, the fat replica may comprise one or more animal fats, although at least some of the animal fats may be produced artificially, e.g., using in vitro techniques such as those described herein. For example, the fat may contain fat cells grown within a bioreactor or other cell culture systems. Even though the fat cells may have originated from an animal, the bulk of the fat cells forming the fat replica may be grown or cultured in an in vitro setting, rather than naturally as part a living animal.

The fat replica may be produced to have any of a variety of characteristics. For example, the fat replica may be present within a cultivated meat product, or other animal-derived product, and it may be desirable for the fat replica to replicate certain characteristics of naturally occurring fat. These may include, for example, content, textures, tastes, mechanical properties, rheological properties (e.g., elastic modulus, loss modulus, etc.), etc. In addition, in some embodiments, the fat replica may include certain types of fat that are desirable or beneficial. For instance, the fat replica may be enriched in certain types of fat that are perceived to be desirable for a particular application. For instance, the fat replica may be enriched or predominantly contain plant-derived fats, saturated or unsaturated fats, or the like.

Thus, in some embodiments, the fat may include an animal fat, a plant fat, or both. In certain embodiments, the fat may include saturated fat, unsaturated fat, or both. In one set of embodiments, a fat replica may comprise non-human fat cells, such as adipose cells, adipose progenitor cells, etc., and/or other types of fat cells. The cells may come from any suitable animal, such as a chicken, a cow, a pig, a sheep, a goat, a deer, a fish, a duck, a turkey, a shrimp, or the like. In some embodiments, the cells may be seeded onto microcarriers, or other scaffolding material, e.g., as discussed herein. In certain cases, the cells may be cultured in a bioreactor or other in vitro cell culture system, such as described herein, to form a fat replica. In certain embodiments, for example, the cells are grown in serum, and induced to differentiate.

In some embodiments, the fat may be present within the fat replica as an emulsion. For example, the fat may be contained within fat particles in the fat replica. In some cases, the fat may be dispersed (for example, homogenously) within the emulsion. In one set of embodiments, for example, fat particles may be formed by mixing fat with non-human blood plasma, which are suspended as an emulsion. The non-human blood plasma may come from any suitable source. As non-limiting examples, the plasma may arise from the blood of an animal, such as a chicken, a cow, a pig, a sheep, a goat, a deer, a fish, a duck, a turkey, a shrimp, etc. The plasma may arise from the same, or a different type of animal than the fat or fat cells that may be present.

In addition, in certain embodiments, a surfactant may be used to disperse the fat and/or stabilize the emulsion. Non-limiting examples of surfactants include phospholipids, monoglyercols, diglycerols, propylene glycol monoesters, lactylate esters, polyglycerol esters, sorbitan esters, ethoxylated esters, succinate esters, fruit acid esters, acetylated monoglycerols, acetylated diglycerols, phosphate monoglycerols, phosphate diglycerols, sucrose esters, etc. For example, a surfactant may be mixed with animal cells and/or non-human blood plasma to form an emulsion.

Non-human blood plasma may be edible. In some cases, the non-human blood plasma may contain fibrinogen, which can be used to form fibrin hydrogels as discussed herein, for example, by the addition of thrombin or calcium. In some embodiments, the non-human blood plasma may be concentrated or diluted, for example, to increase or decrease the crosslinking density of the hydrogel. Accordingly, in another set of embodiments, the fat replica may comprise a hydrogel. Fat may be present within the hydrogel, e.g., suspended within a fluid contained within the hydrogel, e.g., as an emulsion and/or present as fat particles, dissolved or suspended within the fluid, etc. Such a system may form a fat replica that can be used within a cultivated meat product, or other animal-derived product such as those described herein.

In one set of embodiments, the hydrogel may be formed, at least in part, by fibrin. Fibrin is a fibrous protein involved in the clotting of blood. It can be formed, for example, by exposing a non-human blood plasma to a clotting agent. For instance, the protease inhibitor thrombin is able to act on fibrinogen, causing the fibrinogen to form fibrin and thereby form a clot. The clot may be edible. Other techniques can also be used to crosslink fibrin in certain cases, e.g., artificially, rather than causing the clotting process to occur. For example, in one set of embodiments, calcium may be added to cause fibrin to crosslink. Thus, in certain embodiments, a fat replica may comprise a hydrogel comprising fibrin, e.g., that has been clotted to form the hydrogel. In other embodiments, the fibrin may not be clotted, but may form a hydrogel, e.g., by inducing crosslinking of the fibrin, for example, chemically.

However, it should be understood that the fat replica is not limited to only fibrin hydrogels. The hydrogel may be formed from other components, in addition to or instead of fibrin. Non-limiting examples of hydrogels include proteins (for example, collagen, gelatin, etc.), polymers (for example, polylactic acid, polyglycolic acid, etc.), and carbohydrates (for example, alginate, hyaluronan, chitosan, cellulose, hydroxymethyl cellulose etc.). In some cases, a hydrogel may be formed by causing components such as these to crosslink, e.g., in the presence of fat, to form a fat replica.

The hydrogels can be non-covalently and/or covalently crosslinked. Non-covalent hydrogels may be stabilized in some embodiments by hydrogen bonding, van der Waals interactions (e.g., hydrophobic interactions), etc. Covalent hydrogels may be formed, for example, by adding a crosslinking agent, bearing a first coupling group, to a crosslinkable material, bearing a second coupling group. The coupling groups can be any functional groups known to those of skill in the art that together form a covalent bond, for example, under mild reaction conditions or physiological conditions. Examples of coupling groups include, but are not limited to, maleimides, N-hydroxysuccinimide (NHS) esters, carbodiimides, hydrazide, pentafluorophenyl (PFP) esters, phosphines, hydroxymethyl phosphines, psoralen, imidoesters, pyridyl disulfide, isocyanates, vinyl sulfones, alpha-haloacetyls, aryl azides, acyl azides, alkyl azides, diazirines, benzophenone, epoxides, carbonates, anhydrides, sulfonyl chlorides, cyclooctynes, aldehydes, and sulfhydryl groups, etc. In some embodiments, coupling groups may include free amines (—NH₂), free sulfhydryl groups (—SH), free hydroxide groups (—OH), carboxylates, hydrazides, alkoxyamines, etc. In some embodiments, a coupling group can be a functional group that is reactive toward sulfhydryl groups, such as maleimide, pyridyl disulfide, or a haloacetyl.

In certain embodiments, the fat replica may be formed by seeding fat cells such as adipose cells, or adipose progenitor cells, onto microcarriers, and culturing them in a bioreactor to form the fat replica. In some cases, fibrin may be used as a scaffolding material, for example, formed as microcarriers. Fibrin is a fibrous protein involved in the clotting of blood. It can be formed by the action of the protease inhibitor thrombin on fibrinogen, which causes it to polymerize and form a clot. Fibrin can be used as a passive scaffolding material in some embodiments. However, in some embodiments, fibrin can specifically bind certain growth factors in the cell culture media that promote cell adhesion, proliferation, and migration. Non-limiting examples include fibronectin, hyaluronic acid, von Willebrand factor, or the like.

In some embodiments, at least 1 wt %, at least 3 wt %, at least 5 wt %, at least 10 wt %, at least 15 wt %, at least 20 wt %, at least 25%, at least 30 wt %, at least 40 wt %, at least 50 wt %, etc. of the product comprises a fat replica. In addition, in certain embodiments, no more than 50 wt %, no more than 40 wt %, no more than 30 wt %, no more than 25 wt %, no more than 20 wt %, no more than 15 wt %, no more than 10 wt %, no more than 5 wt %, no more than 3 wt %, or no more than 1 wt % of the product comprises a fat replica. Combinations of any of these are also possible in certain cases; for example, the product may have between 5 wt % and 10 wt %, between 10 wt % and 25 wt %, between 1 wt % and 20 wt %, etc. of a fat replica.

In addition, it should be understood that combinations of any of the above are also possible in various embodiments. For example, in one embodiment, a meat product may comprise a texturized plant protein, a non-human blood plasma, and a lysate of non-human red blood cells. In another embodiment, a meat product may comprise a texturized plant protein, blood plasma, a cell lysate, a muscle replica, and a fat replica. Other combinations of any of the above, and/or other components such as those described herein are also possible in still other embodiments.

Without wishing to be bound by any theory, in certain embodiments, such cultivated meat products, or other animal-derived products such as are described herein, may offer certain advantages over other products that do not contain any components derived from an animal. Indeed, many such commercially-available products are “animal-free,” and thus, no part of the product is derived from an animal. In contrast, a variety of products described herein are actually derived from animals, for example, from the blood of an animal, and thus are not animal-free. However, products such as cultivated meat products that include animal-derived components in addition to texturized plant proteins may be more realistic than comparable animal-free meat products, due to the fact that the product is, in part, derived from an animal, and thus offers the actual taste of the animal in the product. However, as discussed herein, due to the way the cultivated meat product (or other animal-derived product) is produced, including the addition texturized plant proteins (which are not produced by killing an animal), and/or the addition of components that can be derived from the blood of an animal, e.g., without killing the animal, such products may be more realistic in features such as taste, texture, etc., while not necessarily being produced through the killing of an animal. However, in other embodiments, it should be understood that such components can be produced from animals that are slaughtered.

The cultivated meat product (or other animal-derived product) may be of any size or mass. For instance, the product may have a mass of at least 10 g, at least 25 g, at least 50 g, at least 100 g, at least 300 g, at least 1 kg, etc. In addition, the product may have any number of additional components, such as blood plasma, cell lysates, muscle replicas, microcarriers, fat replicas, emulsions, hydrogels, etc., e.g., as discussed herein, in any suitable amounts of concentrations, e.g., in addition to texturized plant proteins. For example, each of the components may independently be present at at least 10 wt %, at least 20 wt %, at least 30 wt %, at least 40 wt %, at least 50 wt %, at least 60 wt %, at least 70 wt %, at least 80 wt %, at least 90 wt %, etc., and/or no more than 95 wt %, no more than 90 wt %, no more than 80 wt %, no more than 70 wt %, no more than 60 wt %, no more than 50 wt %, no more than 40 wt %, no more than 30 wt %, no more than 20 wt %, no more than 10 wt %, etc. of the product.

In addition, some embodiments of the present disclosure are directed toward repeated blood collection from non-human animals, for example, to obtain blood, blood serum, blood plasma, red blood cells, cell lysates, muscle replicas, fat replicas, or other components, etc. As mentioned, this can be accomplished without killing the animal. For instance, in some embodiments, blood may be withdrawn from an animal at spaced intervals, so as to allow the animal time to recover and produce new blood. For instance, blood may be withdrawn from the animal every 2 weeks, every 4 weeks, every 6 weeks, every 2 months, or the like. The blood draws may each be processed, for example, as discussed herein. For example, the blood may be used to form a product such as a cultivated meat product. In this way, blood can be obtained in certain embodiments in a sustainable and cost-effective manner, without killing the animal. This usage may result, in certain embodiments, in the reduction in carbon emissions, water use, land use, etc. A variety of animals may be used, such as cows, sheep, pigs, goats, chickens, deer, camel, bison, ducks, fish, turkeys, shrimps, etc., or other animals such as those described herein. See, e.g., Int. Pat. Apl. Ser. No. PCT/US22/19631, entitled “Methods and Systems of Producing Products Such as Animal Derived Products,” filed on Mar. 9, 2022, the disclosure of which is hereby incorporated by reference.

Thus, some embodiments are directed at sustainably procuring blood components or other components for producing cultivated meat products or other animal-derived products, for example, in a bioreactor. In some embodiments, blood can be collected from non-human animals, for example, to obtain platelets, platelet-rich plasma, white blood cells, red blood cells, etc. For instance, repeated blood collection from non-human animals may be used to obtain immune cells, or the like.

In some embodiments, certain animal derived blood products, such as platelets, red blood cells, etc., may be safely harvested from different animals such as bovines, equines and canines. Thus, in some embodiments, blood components, such as platelets and red blood cells, may be repeatedly harvested from animals, e.g., without causing health issues to the animals. For example, platelets and platelet rich plasma can be harvested at least 1×, at least 2×, at least 3×, at least 4×, etc., every 30 days, or at other rates such as those described herein. In this way, blood components may be sustainably harvested and used, for example, for the production of cultivated meat products or other animal-derived products without slaughtering the animal and/or without the adverse environmental impacts of raising animals for slaughter. However, it should be understood that in other embodiments, whole blood and/or blood components may be harvested from animals that are slaughtered.

In one set of embodiments, one or more blood draws may be withdrawn or taken from a non-human living animal. The blood may be processed in some cases to isolate or prepare various components, e.g., plasma, red blood cells etc., using any suitable technique, e.g., centrifugation, apheresis, or the like.

In some embodiments, a plurality of blood draws from a non-human living animal may be performed over a period of time. For example, in some cases, blood may be withdrawn from a donor animal at spaced intervals, which may be regular or irregular. Between blood draws, the animal can recover and produce new blood. Any suitable interval may be used. For example, the blood may be withdrawn from the animal every 2 weeks, every 4 weeks, every 6 weeks, every 2 months, or the like. In addition, in some cases, blood may be withdrawn from an animal at a period of greater than 1 day, greater than 2 days, greater than 3 days, greater than 4 days, greater than 5 days, greater than 6 days, greater than 1 week, greater than 2 weeks, greater than 3 weeks, greater than 4 weeks, greater than 5 weeks, greater than 6 weeks, greater than 7 weeks, greater than 8 weeks, greater than 9 weeks, greater than 10 weeks, greater than 11 weeks, greater than 12 weeks, greater than 13 weeks, greater than 14 weeks, greater than 15 weeks, greater than 16 weeks, etc. In certain embodiments, the period may be no more than 16 weeks, no more than 15 weeks, no more than 14 weeks, no more than 13 weeks, no more than 12 weeks, no more than 11 weeks, no more than 10 weeks, no more than 9 weeks, no more than 8 weeks, no more than 7 weeks, no more than 6 weeks, no more than 5 weeks, no more than 4 weeks, no more than 3 weeks, no more than 2 weeks, no more than 1 week, no more than 6 days, no more than 5 days, no more than 4 days, no more than 3 days, no more than 2 days, etc. Combinations of any of these are also possible. For example, the period may be between 3 weeks and 5 weeks, between 6 days and 2 weeks, or the like. As mentioned, in some case, the blood withdrawal may be relatively irregular, but the average period of withdraws may, in some embodiments, be within these ranges.

In some embodiments, the first blood draw and the second blood draw are separated by at least 24 hours. As an example, in some embodiments, a blood draw may be withdrawn from an animal, and after a suitable interval, e.g., 24 hours, an additional blood draw may be withdrawn from the animal. This cycle can also be repeated any suitable number of times. In addition, in some embodiments, the first blood draw and the second draw may be separated by at least 1 day, at least 2 days, at least 3 days, at least 4 days, at least 5 days, at least 6 days, at least 7 days, at least 14 days, at least 21 days, at least 28 days, at least 5 weeks, at least 6 weeks, at least 7 weeks, at least 8 weeks, etc. The blood draws may be processed, for example, as discussed herein. For example, the blood may be used to isolate platelets and/or platelet-rich plasma.

In addition, the animal may also be subjected to second, third, etc. blood draws, e.g., at spaced intervals such as discussed herein. The blood withdrawn in each draw may be treated in the same way, or in different ways, depending on the application. In some embodiments, the animal remains alive between blood draws, and can recover and produce new blood. In this way, although blood production from the animal occurs, the animal is not slaughtered in order for blood production from the animal to continue.

Thus, in some embodiments, animals can be raised or “farmed” for the purpose of being sources of components such as those discussed herein. Contemplated in certain embodiments are methods by which farm animals can be raised for multiple purposes including, for example, for the harvesting of blood products such cows, sheep and goats producing milk and sheep producing wool.

Some embodiments are directed to raising a non-living animal blood donor on a farm, such as a regenerative farm. Herein, the term regenerative farming is synonymous with regenerative agriculture, agricultural farming, regenerative growers, regenerative grazing, and the like. As will be appreciated by those of skill in the art, the majority of the food consumed around the world is grown through varying methods of intensive industrial agriculture, a form of modern farming that became prevalent during the Industrial Revolution. However, industrial agriculture has been linked to problems such as (i) diminished biodiversity among plants and animals, (ii) soil, water, and air pollution, (iii) soil erosion, (iv) unsustainable rates of water consumption, (v) development of chronic disease, cancers, foodborne pathogens, pesticides, and high-speed meat production, (vi) antibiotic resistance, and (vii) a lack of nutritional value in food. Regenerative farming is a holistic approach to agriculture that focuses on the interconnection of farming systems and the ecological system as a whole.

Regenerative farming may involve the integration of animals into the farm as much as possible. For example, regenerative grazing is one practice of regenerative farming that builds soil health by managing livestock on perennial and annual forages. One outcome of current industrial agricultural practices is monocropping, in which a single crop, e.g., wheat, is grown on the same piece of land year after year. Such practices strip the soil of its nutrients and requires hauling manure at additional cost or importing synthetic fertilizers from off the farm to support continued growth of the crops. However, proper management of livestock using, for example, regenerative grazing practices, can provide the manure needed to replace nutrients to the soil, reducing the need for fertilizers, and increasing soil organic matter. For example, healthy soils capture large amounts of carbon and water and reduce the amount of polluted runoff.

In some cases, regenerative farming may involve practices of reusing and recycling frequently, e.g., wherever possible. As such, in some embodiments, a single animal can be used for multiple purposes, such as blood donation and milk production. For example, a cow can be used to donate bovine blood for producing cultivated meat products and milk; a sheep may also be used to donate blood for cultivated meat production and wool.

In some embodiments, regenerative farming may involve sustainability. In some embodiments, regenerative farming practices may lower the economic hurdles associated with large scale commercialization of cultivated meats by providing a cell growth media in a sustainable manner. Currently, fetal bovine serum is used as the standard growth supplement for most cell culture media. Fetal bovine serum is typically obtained from a bovine fetus, via a closed collection system such as at a slaughterhouse and is not intended to be manufactured in a sustainable or cost-effective way.

Some embodiments are directed toward methods of quantifying the decrease in the carbon footprint of the regenerative farm. For example, in some embodiments, the method comprises harvesting whole blood from a non-human animal, separating the whole blood into components, and using components to produce a product, e.g., as discussed herein. Other embodiments are directed toward methods comprising raising a non-human living animal on a regenerative farm, withdrawing a plurality of blood draws from the non-human living animal, and forming a plurality of products using the plurality of blood draws. In some embodiments, the regenerative farm has a net zero carbon footprint.

In other embodiments, the method comprises grazing a non-human animal on a plurality of pastures such that the non-human animal increases the carbon content of each of the pastures by at least 50 mass %, at least 60 mass %, or at least 70 mass % due to its waste, relative to identical pastures in the absence of the non-human animal, withdrawing a plurality of blood draws from the non-human animal, and using the blood draws to produce a product. Rotating the field that the non-human animals graze, also known as regenerative grazing, is a regenerative farming practice that uses the manure produced by grazing animals to fertilize and maintain soil health. In some embodiments, the non-human living animals comprise multiple different species, such as for example, cow, goat, sheep, horses, etc. In some embodiments, regenerative grazing comprises rotating the grazing of the non-human animals on a plurality of pastures. In some embodiments, the pastures may be rotated every day, every second day, every third day, every fourth day, every fifth day, every sixth day, every seventh day, every 2 weeks, every 4 weeks, every 8 weeks, every 16 weeks, every 32 weeks, or every 52 weeks. In some embodiments, rotational grazing may reduce the greenhouse gas emissions by between 10 mass % and 50 mass %, relative to identical pastures in the absence of rotational grazing.

The following are each incorporated herein by reference in their entireties: U.S. Provisional Patent Application Ser. No. 63/159,403, filed Mar. 10, 2021, entitled “Constructs for Meat Cultivation and Other Applications”; U.S. Provisional Patent Application Ser. No. 63/279,617, filed Nov. 15, 2021, entitled “Constructs Comprising Fibrin or Other Blood Products for Meat Cultivation and Other Applications”; U.S. Provisional Patent Application Ser. No. 63/279,631, filed Nov. 15, 2021, entitled, “Methods and Systems of Preparing Cultivated Meat from Blood or Cellular Biomass”; U.S. Provisional Patent Application Ser. No. 63/279,642, filed Nov. 15, 2021, entitled, “Systems and Methods of Producing Fat Tissue for Cell-Based Meat Products”; U.S. Provisional Patent Application Ser. No. 63/279,644, filed Nov. 15, 2021, entitled “Production of Heme for Cell-Based Meat Products”; U.S. Provisional Patent Application Ser. No. US 63/300,577, filed Jan. 18, 2022, entitled “Animal-Derived Antimicrobial Systems and Methods”; U.S. Provisional Patent Application Ser. No. 63/164,397, filed Mar. 22, 2021, entitled “Growth Factor for Laboratory Grown Meat”; U.S. Provisional Patent Application Ser. No. 63/164,387, filed Mar. 22, 2021, entitled, “Methods of Producing Animal Derived Products”; U.S. Provisional Patent Application Ser. No. 63/314,171, filed Feb. 25, 2022, entitled “Growth Factors for Laboratory Grown Meat and Other Applications”; and U.S. Provisional Patent Application Ser. No. 63/314,191, filed Feb. 25, 2022, entitled “Methods and Systems of Producing Products Such as Animal Derived Products.” In addition, U.S. Provisional Patent Application Ser. No. 63/330,814, filed Apr. 24, 2022, entitled “Systems and Methods for Protein Recovery from Cell Culture Media,” is incorporated herein by reference in its entirety.

In addition, U.S. Provisional Patent Application Ser. No. 63/330,814, filed Apr. 14, 2022, entitled “Systems and Methods for Protein Recovery from Cell Culture Media,” and U.S. Provisional Patent Application Ser. No. 63/337,554, filed Apr. 14, 2022, entitled “Systems and Methods of Preparing Cultivated Meat and Meat Analogs from Plant-Based Proteins” are each incorporated herein by reference in its entirety.

The following examples are intended to illustrate certain embodiments of the present disclosure, but do not exemplify the full scope of the disclosure.

### Example 1

This example describes a production method in accordance with one embodiment. In this example, a plant-based meat product was produced by mixing textured soy and textured pea protein with a lysate of non-human red blood cells. A muscle replica comprising plant-based protein was prepared, based on methods described in Int. Pat. Apl. Ser. No. PCT/US2022/019594, filed on Mar. 9, 2022, entitled “Constructs Comprising Fibrin or Other Blood Products for Meat Cultivation and Other Applications,” incorporated herein by reference in its entirety.

Fresh whole blood was obtained from a living cow (without killing the cow) and the red blood cells were separated from the whole blood by centrifugation at 150 g for 25 minutes at 4° C. The isolated red blood cells were then lysed using distilled water to a final concentration of 20% v/v. Although non-lysed red blood cells could also be used as a colorant in other embodiments, in these examples, higher concentrations of unlysed red blood cells were used to obtain the same color profile as lysed red blood cells (see, e.g., Int. Pat. Apl. Ser. No. PCT/US2022/019615, filed on Mar. 9, 2022, entitled “Production of Heme for Cell-Based Meat Products,” incorporated herein by reference in its entirety). After the addition of the red blood cells, and providing sufficient time for the red blood cells to diffuse into the dried textured plant protein, the 20% v/v of plasma separated from whole blood was then added to the mixture as a binder and nourishing additive.

In some embodiments, a red colorant was obtained by mixing a 3% (vol/vol) lysate of non-human red blood cells with 77% textured plant protein and 20% fat replica prepared as discussed in Int. Pat. Apl. Ser. No. PCT/US2022/019609, filed Mar. 9, 2022, entitled, “Systems and Methods of Producing Fat Tissue for Cell-Based Meat Products,” incorporated herein by reference in its entirety. These were mixed together to form a cultivated meat product resembling ground beef. In some embodiments, the cultivated meat product may be mixed with a binding agent and a crosslinking agent such as transglutaminase. The binding agent may be non-human plasma (20% vol/vol) and the crosslinking agent may be calcium chloride (0.1% w/v) or thrombin. In some embodiments, the cultivated meat product may be cut and pressed to adjust the moisture content. In some embodiments, the cultivated meat product may be extruded to form fibers.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A meat product, comprising:
a texturized plant protein;
non-human blood plasma; and
a lysate of non-human red blood cells.

2. The meat product of claim 1, wherein the texturized plant protein is an extruded protein.

3. The meat product of any one of claim 1 or 2, wherein the texturized plant protein is a fiber-spun protein.

4. The meat product of any one of claims 1-3, wherein the texturized plant protein comprises texturized soy protein.

5. The meat product of any one of claims 1-4, wherein the texturized plant protein comprises texturized pea protein.

6. The meat product of any one of claims 1-5, wherein the texturized plant protein comprises texturized corn protein.

7. The meat product of any one of claims 1-6, wherein the texturized plant protein comprises texturized lentil protein.

8. The meat product of any one of claims 1-7, wherein the texturized plant protein comprises texturized peanut protein.

9. The meat product of any one of claims 1-8, wherein the texturized plant protein comprises texturized mycoprotein protein.

10. The meat product of any one of claims 1-9, wherein the texturized plant protein comprises texturized quinoa protein.

11. The meat product of any one of claims 1-10, wherein the texturized plant protein comprises texturized chickpea protein.

12. The meat product of any one of claims 1-11, wherein the texturized plant protein comprises texturized cottonseed protein.

13. The meat product of any one of claims 1-12, wherein the texturized plant protein comprises texturized wheat protein.

14. The meat product of any one of claims 1-13, wherein the texturized plant protein comprises texturized oat protein.

15. The meat product of any one of claims 1-14, wherein the non-human blood plasma comprises fibrin.

16. The meat product of any one of claims 1-15, wherein the non-human blood plasma comprises fibrinogen.

17. The meat product of any one of claims 1-16, wherein the non-human blood plasma is crosslinked.

18. The meat product of any one of claims 1-17, wherein the non-human blood plasma comprises cow blood plasma.

19. The meat product of any one of claims 1-18, wherein the non-human blood plasma comprises pig blood plasma.

20. The meat product of any one of claims 1-19, wherein the non-human blood plasma comprises chicken blood plasma.

21. The meat product of any one of claims 1-20, wherein the non-human blood plasma comprises turkey blood plasma.

22. The meat product of any one of claims 1-21, wherein the non-human blood plasma and the lysate of non-human red blood cells are from different species.

23. The meat product of any one of claims 1-22, wherein the non-human blood plasma and the lysate of non-human red blood cells are from the same species.

24. The meat product of any one of claims 1-23, wherein the non-human blood plasma and the lysate of non-human red blood cells are from the same animal.

25. The meat product of any one of claims 1-24, wherein the non-human blood plasma is harvested from a living animal by apheresis.

26. The meat product of any one of claims 1-25, wherein the lysate of non-human red blood cells is harvested from a living animal by apheresis.

27. The meat product of any one of claims 1-26, further comprising a fat emulsion.

28. The meat product of claim 27, wherein the fat emulsion is present within a fat replica.

29. The meat product of any one of claim 27 or 28, wherein the fat emulsion comprises the non-human blood plasma.

30. The meat product of any one of claims 27-29, wherein the fat emulsion comprises a hydrogel.

31. The meat product of any one of claims 27-30, wherein the hydrogel comprises an alginate.

32. The meat product of any one of claims 27-31, wherein the hydrogel comprises a chitosan.

33. The meat product of any one of claims 27-32, wherein the fat emulsion comprises animal fat.

34. The meat product of any one of claims 27-33, wherein the fat emulsion comprises plant-based fat.

35. The meat product of any one of claims 27-34, wherein the fat emulsion comprises cultivated adipose cells.

36. The meat product of any one of claims 1-35, further comprising microcarriers.

37. The meat product of claim 36, comprising non-human animal cells on the microcarriers.

38. The meat product of any one of claim 36 or 37, wherein the non-human animal cells are cultivated on the microcarriers.

39. The meat product of any one of claims 36-38, wherein the microcarriers comprise a polymer.

40. The meat product of any one of claims 36-39, wherein the microcarriers comprise fibrin.

41. The meat product of any one of claims 36-40, wherein the microcarriers comprise carbohydrate.

42. The meat product of any one of claims 36-41, wherein the microcarriers comprise a polysaccharide.

43. The meat product of any one of claims 36-42, wherein the microcarriers comprise a protein.

44. The meat product of any one of claims 36-43, wherein the microcarriers comprise a plant-based material.

45. The meat product of any one of claims 36-44, wherein the microcarriers have a largest dimension of no more than 50 mm.

46. The meat product of any one of claims 1-45, further comprising non-human cells.

47. The meat product of any one of claims 1-46, wherein the non-human cells comprise myoblasts.

48. The meat product of any one of claims 1-47, wherein the non-human cells comprise fibroblasts.

49. The meat product of any one of claims 1-48, wherein the meat product has a mass of at least 10 g.

50. The meat product of any one of claims 1-49, wherein at least 50 wt % of the meat product comprises the texturized plant protein.

51. The meat product of any one of claims 1-50, wherein at least 60 wt % of the meat product comprises the texturized plant protein.

52. The meat product of any one of claims 1-51, wherein at least 70 wt % of the meat product comprises the texturized plant protein.

53. The meat product of any one of claims 1-52, wherein no more than 25 wt % of the meat product comprises non-human blood plasma.

54. The meat product of any one of claims 1-53, wherein no more than 5 wt % of the meat product comprises the lysate of non-human red blood cells.

55. A meat product, comprising:
a ground beef replica comprising a texturized plant protein, non-human muscle cells, non-human blood plasma, and a lysate of non-human red blood cells.

56. The meat product of claim 55, wherein the ground beef replica has a mass of at least 10 g.

57. The meat product of any one of claim 55 or 56, wherein the texturized plant protein is an extruded protein.

58. The meat product of any one of claims 55-57, wherein the texturized plant protein is a fiber-spun protein.

59. The meat product of any one of claims 55-58, wherein the non-human red blood cells comprise cow red blood cells.

60. The meat product of any one of claims 55-59, wherein the non-human blood plasma is harvested from a living animal by apheresis.

61. The meat product of any one of claims 55-60, wherein the lysate of non-human red blood cells is harvested from a living animal by apheresis.

62. A meat product, comprising:
a texturized plant protein; and
non-human blood plasma.

63. The meat product of claim 62, wherein the texturized plant protein is an extruded protein.

64. The meat product of any one of claim 62 or 63, wherein the texturized plant protein is a fiber-spun protein.

65. The meat product of any one of claims 62-64, wherein the non-human blood plasma comprises cow blood plasma.

66. A meat product, comprising:
a texturized plant protein; and
a fat replica comprising a fat emulsion and non-human blood plasma.

67. The meat product of claim 66, wherein the texturized plant protein is an extruded protein.

68. The meat product of any one of claim 66 or 67, wherein the texturized plant protein is a fiber-spun protein.

69. The meat product of any one of claims 66-68, wherein the non-human blood plasma comprises cow blood plasma.

70. A meat product, comprising:
a texturized plant protein;
microcarriers comprising fibrin; and
non-human cells on the microcarriers.

71. The meat product of claim 70, wherein the texturized plant protein is an extruded protein.

72. The meat product of any one of claim 70 or 71, wherein the texturized plant protein is a fiber-spun protein.

73. The meat product of any one of claims 70-72, wherein the non-human cells comprises cow cells.

74. The meat product of any one of claims 70-73, wherein the non-human cells are cultivated on the microcarriers.

75. A meat product, comprising:
a texturized plant protein; and
a muscle replica comprising microcarriers and non-human muscle cells.

76. The meat product of claim 75, wherein the texturized plant protein is an extruded protein.

77. The meat product of any one of claim 75 or 76, wherein the texturized plant protein is a fiber-spun protein.

78. The meat product of any one of claims 75-77, wherein the non-human muscle cells comprise cow muscle cells.

79. The meat product of any one of claims 75-78, wherein the non-human muscle cells are cultivated on the microcarriers.

80. A meat product, comprising:
a texturized plant protein; and
a hydrogel comprising crosslinked non-human blood plasma.

81. The meat product of claim 80, wherein the texturized plant protein is an extruded protein.

82. The meat product of any one of claim 80 or 81, wherein the texturized plant protein is a fiber-spun protein.

83. The meat product of any one of claims 80-82, wherein the non-human blood plasma comprises cow blood plasma.

84. A method, comprising:
withdrawing a blood sample from a non-human animal;
processing the blood sample to produce non-human blood plasma and a lysate of non-human red blood cells; and
producing a product comprising a mixture of the non-human blood plasma, the lysate of non-human red blood cells, and texturized plant protein.

85. The method of claim 84, wherein the product is a cultivated meat product.

86. The method of any one of claim 84 or 85, wherein the non-human red blood cells comprise cow red blood cells.

87. A method, comprising:
mixing a texturized plant protein and non-human blood plasma to produce a product.

88. The method of claim 87, wherein the product is a meat product.

89. The method of any one of claim 87 or 88, wherein the non-human blood plasma comprises cow blood plasma.

90. A method, comprising:
mixing a texturized plant protein and a lysate of non-human red blood cells to produce a product.

91. The method of claim 90, wherein the product is a meat product.

92. The method of any one of claim 90 or 91, wherein the non-human red blood cells comprises cow red blood cells.

93. A meat product, comprising:
a texturized plant protein; and
non-human blood.

94. The meat product of claim 93, wherein the non-human blood arises from a slaughtered animal.

95. The meat product of any one of claim 93 or 94, wherein the non-human blood arises from an animal that was not slaughtered.

96. The meat product of any one of claims 93-95, wherein the non-human blood is cow blood.

97. The meat product of any one of claims 93-96, wherein the meat product has a mass of at least 10 g.

98. The meat product of any one of claims 93-97, wherein the meat product is a ground beef replica.

99. A meat product, comprising:
a texturized plant protein; and
non-human red blood cells.

100. The meat product of claim 99, wherein the non-human red blood cells arise from a slaughtered animal.

101. The meat product of any one of claim 99 or 100, wherein the non-human red blood cells arise from an animal that was not slaughtered.

102. The meat product of any one of claims 99-101, wherein the non-human red blood cells are cow red blood cells.

103. The meat product of any one of claims 99-102, wherein the meat product has a mass of at least 10 g.

104. The meat product of any one of claims 99-103, wherein the meat product is a ground beef replica.

105. A meat product, comprising:
a texturized plant protein; and
non-human blood plasma.

106. The meat product of claim 105, wherein the non-human blood plasma arises from a slaughtered animal.

107. The meat product of any one of claim 105 or 106, wherein the non-human blood plasma arises from an animal that was not slaughtered.

108. The meat product of any one of claims 105-107, wherein the non-human blood plasma is cow blood plasma.

109. The meat product of any one of claims 105-108, wherein the meat product has a mass of at least 10 g.

110. The meat product of any one of claims 105-109, wherein the meat product is a ground beef replica.

111. A meat product, comprising:
a texturized plant protein;
non-human blood plasma; and
non-human red blood cells.

112. The meat product of claim 111, wherein the non-human red blood cells and the non-human blood plasma are from the same species.

113. The meat product of any one of claim 111 or 112, wherein the non-human red blood cells and the non-human blood plasma are from the same animal.

114. The meat product of any one of claims 111-113, wherein the non-human red blood cells arise from a slaughtered animal.

115. The meat product of any one of claims 111-114, wherein the non-human red blood cells arise from an animal that was not slaughtered.

116. The meat product of any one of claims 111-115, wherein the non-human red blood cells are cow red blood cells.

117. The meat product of any one of claims 111-116, wherein the meat product has a mass of at least 10 g.

118. The meat product of any one of claims 111-117, wherein the meat product is a ground beef replica.

119. A method, comprising:
mixing a texturized plant protein, non-human blood plasma, and a lysate of non-human red blood cells to produce a meat product.

120. The meat product of claim 119, wherein the texturized plant protein is an extruded protein.

121. The meat product of any one of claim 119 or 120, wherein the texturized plant protein is a fiber-spun protein.

122. The meat product of any one of claims 119-121, wherein the non-human red blood cells comprise cow red blood cells.
